(19) United States
(12) Patent Application Publication
(10) Pub. No.: US 2019/0070411 A1
(43) Pub. Date: Mar. 7, 2019

(54) ACTIVE IMPLANTABLE MEDICAL DEVICE FOR COMBINED TREATMENT OF CARDIAC RHYTHM AND OF RESPIRATORY RHYTHM

(71) Applicant: SORIN CRM SAS, Clamart (FR)

(72) Inventor: Willy Regnier, Longjumeau (FR)

(21) Appl. No.: 16/084,939

(22) PCT Filed: Mar. 17, 2017
(86) PCT No.: PCT/EP2017/056419

§ 371 Date: Sep. 13, 2018

(30) Foreign Application Priority Data
Mar. 18, 2016 (FR) 1652297

(51) Int. Cl. A61N 1/36; A61N 1/365; A61N 1/05; A61N 1/372; A61N 1/375

(52) U.S. Cl. CPC A61N 1/3611; A61N 1/365; A61N 1/36139; A61N 1/37512; A61N 1/0551; A61N 1/37217; A61N 1/3756; A61N 1/056

(57) ABSTRACT

This device comprises an autonomous subcutaneous unit with a leak-tight body, incorporating a control circuit for the cardiac rhythm, a control circuit for the respiratory rhythm, and a combined operating circuit for operating the control circuits for cardiac rhythm and respiratory rhythm. It additionally comprises at least one cardiostimulation microprobe with at least one electrode implanted in or on the myocardium, and at least one neurostimulation microprobe with at least one electrode to be implanted on or near a nerve of the patient. The device overall is in the form of an autonomous hybrid capsule integrating, within one and the same assembly, the autonomous unit, the cardiostimulation probe and the neurostimulation probes, without an electrical connector between the electronic circuits and the microprobes, and each of the probes is a continuation of the leak-tight body of the autonomous unit at one side of the latter.

### CROSS-REFERENCE TO RELATED PATENT APPLICATIONS

This application is a 371 U.S. National application of International Application No. PCT/EP2017/056419, filed Mar. 17, 2017, which claims the benefit of and priority to French Patent Application No. 1652297, Mar. 18, 2016, which is incorporated herein by reference in its entirety.

### BACKGROUND

The invention relates to “active implantable medical devices” as defined by Council of the European Communities Directive 90/385/EEC of 20 Jun. 1990, and more specifically to detecting electric potentials produced by organs and/or electrically stimulating such organs.

It relates more specifically to a device for combined stimulation of the myocardium and of the peripheral nervous system, indicated, for example, for patients suffering from cardiac rhythm disorders associated with pathologies known as “sleep apnea” (SA) or “sleep apnea syndrome (SAS). Sleep apnea, be it obstructive or central, is a pathology that can give rise to certain disorders such as excessive daytime sleepiness (EDS), arrhythmias, and hypertension (high blood pressure), and that can worsen the general condition of patients suffering from heart failure. Indeed, central sleep apnea, which has neurological causes and does not result from an obstruction to breathing in, may appear as a consequence of heart failure.

It is therefore advantageous, in such patients, to treat the cardiac pathology and the respiratory pathology jointly by using a therapy of stimulating the peripheral nervous system with application of electric pulses delivered by a lead having its active portion implanted on a nerve or close to a nerve (such therapy being referred to below as “neurostimulation”), combined with a therapy of anti-bradycardia stimulation or of resynchronization of the myocardium with application of depolarization pulses delivered by one or more leads having their active portions implanted in the ventricular and/or atrial cavities (such therapy being referred to below as “cardiostimulation”).

Document US 2008/0208282 A1 (U.S. Pat. No. 8,909,341 B2) describes such device for joint stimulation of the myocardium and of the nervous system for treating sleep apnea.

That device comprises a generator connected to i) cardiac detection/stimulation leads implanted in both ventricles, right and left, ii) a lead for stimulating the region innervated by the hypoglossal nerve, so as to modify the sympathovagal balance of the patient by applying low-energy neurostimulation electric pulses delivered in phase with the biventricular stimulation or pacing, and iii) a lead for stimulating the diaphragm by applying neurostimulation electric pulses to the phrenic nerve. The lead for stimulating the diaphragm also acts as a lead for collecting the nerve potentials produced at the rhythm of the breathing movements, thereby enabling the device to synchronize the pulses for stimulating the phrenic nerve with the natural breathing cycle of the patient.

That known device uses conventional technologies, with a generator provided with a connection head receiving the proximal end of each of the four leads, the respective distal ends of which have been implanted at the corresponding stimulation sites. It therefore suffers from the drawbacks specific to conventional stimulators, namely the volume of the generator, the difficulty of reaching the stimulation sites (in particular in the coronary venous system for stimulating the left ventricle), and the need to make provision, in the implantation procedure, for having a step for connecting the four leads to the generator, as well as for having a test step for testing the leads so as to check the conformity of the interaction between each lead and the generator.

### SUMMARY

The object of the invention is to propose a novel device structure for combined treatment of cardiac rhythm and of respiratory rhythm that mitigates that type of limitation suffered by known devices.

To this end, the invention provides an implantable device for combined treatment of cardiac rhythm and of respiratory rhythm of the general type disclosed by above-mentioned US 2008/0208282 A1, i.e. a device that comprises:
an autonomous unit designed to be implanted subcutaneously, said autonomous unit comprising a sealed body receiving electronic circuits and electrical power supply means for powering said electronic circuits, the electronic circuits comprising:
a cardiac rhythm control circuit, comprising a circuit for detecting cardiac potentials and a generator for generating cardiostimulation pulses;
a respiratory rhythm control circuit, comprising a circuit for detecting nerve potentials and a generator for generating neurostimulation pulses; and
a combined control circuit for combined control of the cardiac rhythm control circuit and of the respiratory rhythm control circuit;
at least one cardiostimulation lead, comprising, in a distal active portion, at least one detection and/or stimulation electrode suitable for being implanted in or on the myocardium of a patient wearing the device; and
at least one neurostimulation lead, comprising, in a distal active portion, at least one detection and/or stimulation electrode suitable for being implanted on or close to a nerve of the patient wearing the device.

In a manner characteristic of the invention:
the at least one cardiostimulation lead and the at least one neurostimulation lead are micro-leads each of which comprises at least one micro-cable formed of an electrically conductive core cable connected to a pole of said electronic circuits, with a layer of insulation surrounding the core cable, which is provided with at least one selectively bared or exposed zone provided in the layer of insulation and designed to form said detection and/or stimulation electrode, the diameter of the micro-lead being no more than 1 French (0.33 millimeters (mm)) in its most distal region including said selectively bared or exposed zone;
said device is an autonomous hybrid capsule incorporating in the same set said autonomous unit, said at least one cardiostimulation lead and said at least one neurostimulation lead,

the hybrid capsule being devoid of any electrical connector between the electronic circuits and the cardiostimulation and neurostimulation micro-leads;

and each of the at least one cardiostimulation lead and of the at least one neurostimulation lead extending the sealed body of the autonomous unit at one end thereof.

According to various advantageous subsidiary characteristics:
the at least one cardiostimulation micro-lead and/or the at least one neurostimulation micro-lead is a multi-pole micro-lead comprising a plurality of said electrodes in its distal active portion;
said cardiostimulation micro-lead (120) is a stimulation micro-lead that is suitable for being implanted in the coronary venous system (CVS) for stimulating a left cavity (LV) of the myocardium of the patient wearing the device;
for an application in which the neurostimulation is indirect stimulation of the diaphragm via the left pericardiophrenic nerve by said neurostimulation micro-lead, said neurostimulation micro-lead is a micro-lead suitable for being implanted in the left pericardiophrenic vein, the length between the body of the autonomous unit and the most distal electrode of the neurostimulation micro-lead being from 50 centimeters (cm) to 60 cm;
for an application in which the neurostimulation is direct stimulation of the diaphragm by said neurostimulation micro-lead, said neurostimulation micro-lead is a micro-lead suitable for being implanted in one of the branched vessels of the left pericardiophrenic vein, the length between the body of the autonomous unit and the most distal electrode of the neurostimulation micro-lead being from 60 cm to 70 cm;
the device further comprises a neurodetection micro-lead suitable for being implanted on or close to a nerve of an afferent pathway of the patient wearing the device, and said neurostimulation micro-lead is a micro-lead suitable for being implanted on or close to a nerve of an efferent pathway of the patient wearing the device;
in that situation, said neurodetection micro-lead is advantageously a micro-lead suitable for being implanted in the right pericardiophrenic vein, the length between the body of the autonomous unit and the most distal electrode of the neurodetection micro-lead being from 55 cm to 65 cm;
the electronic circuits of the autonomous unit further comprise transmitter/receiver means for mutual wireless intra-body with intra-body autonomous leadless capsules suitable for being implanted in or on the myocardium of the patient, and interface mans for enabling said transmitter/receiver means to interface with the cardiac rhythm control circuit.

### BRIEF DESCRIPTION OF THE DRAWINGS

An embodiment of the present invention is described below with reference to the accompanying drawings, in which like references designate elements that are identical or functionally similar from one figure to another, and in which:

FIG. 1 is an overall view showing, as implanted, the various elements of the device of the invention, which elements are used in combination in a system further comprising pacemaker leadless capsules for stimulating the right cavities of the heart;

FIG. 2 is a diagrammatic view showing, as not implanted, the elements of the system shown in FIG. 1;

FIG. 3 is an enlarged view of the hybrid capsule of the device of the invention; and

FIG. 4 is a block diagram showing the various elements of the system of the invention and the interactions between the various elements.

### DETAILED DESCRIPTION

An embodiment of the device of the invention is described below.

The invention is described more specifically in the context of an implant making it possible to deliver Cardiac Resynchronization Therapy (CRT) or Bi-Ventricular Pacing (BVP), consisting in continuously monitoring cardiac rhythm and, if necessary, in delivering electric pulses to the heart, thereby making it possible to stimulate the left and right ventricles jointly and continuously so as to resynchronize them. In such a case, anti-bradycardia stimulation or pacing involves monitoring of electric potentials for depolarizing the myocardium, and controlled delivery of pulses jointly to both ventricles. However, this particular situation is not limiting to the invention which, through a some of its aspects, is also applicable to devices of the “single chamber” type where the detection/stimulation concerns the right ventricle only, or of the “double chamber” type where the detection/stimulation concerns the right ventricle and the right atrium only.

In general, the invention relates to a device including at least one cardiostimulation or pacemaker lead, i.e. a lead that is suitable for being implanted in or on the myocardium of a patient wearing the device. In the example shown, the lead is an endovascular lead for detecting/stimulating a left cavity, and in particular the left ventricle, via the coronary venous system, access to which is difficult (approach to the coronary sinus, and navigating through the narrow vessels of the venous system, etc.). This particular situation is the one that takes best advantage of the advantageous characteristics of the device of the invention, but the invention is equally as applicable to a device including a pacemaker lead for stimulating some other cardiac cavity, such as the right ventricle and/or the right atrium, instead of or in addition to a pacemaker lead for stimulating the left ventricle.

Similarly, the invention is described in the context of a set that, in addition to the device of the invention, comprises pacemaker autonomous devices of the leadless capsule type, in particular for stimulating the right cavities of the heart, but those leadless capsules are not part of the device of the invention per se, and the invention may also be implemented without such capsules, or with other means for stimulating the right cavities.

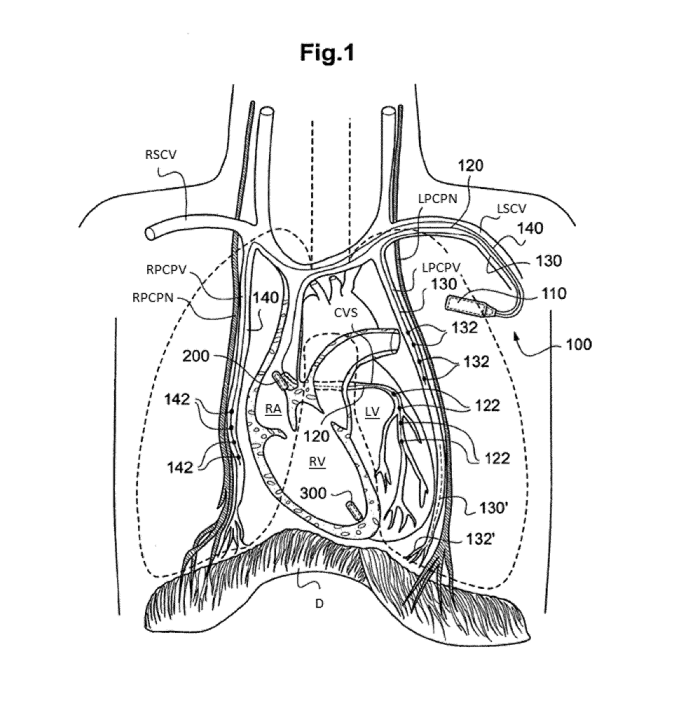

FIG. 1 shows the various elements of the device of the invention, as implanted in the body of a patient, the various organs of the patient being referenced as follows:

LSCV, RSCV: left and right subclavian veins;

LPCPV, RPCPV: left and right pericardiophrenic veins;

LPCPN, RPCPN: left and right pericardiophrenic nerves, which are nerves running along the respective pericardiophrenic veins;

CVS: coronary venous system;

RA, RV, LV: right atrium, right ventricle, left ventricle; and

D: diaphragm.

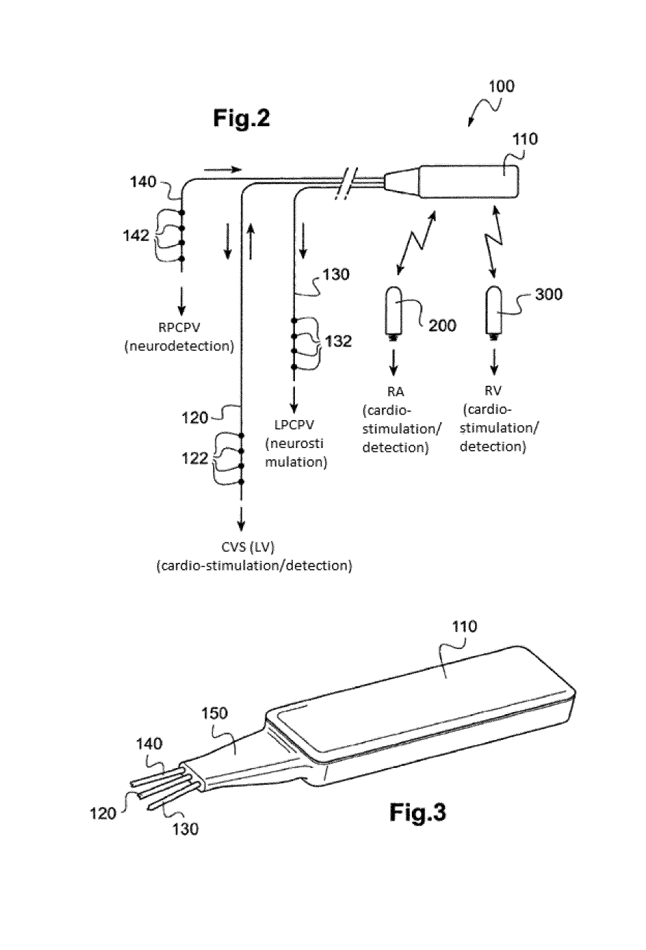

The device 100 of the invention, shown as implanted in FIG. 1, is also shown separately and schematically in FIG. 2. The device 100 comprises a hybrid capsule 110 implanted in a subcutaneous region of the body of the patient.

The term “hybrid capsule” is used to mean a device:
comprising a body of the same shape and of the same configuration as a leadless capsule (see below for what is meant by this term), with a low-consumption electronics architecture, a miniaturized energy source and wireless communications means for communicating with other capsules;
where said body is provided with a lead extending the body of the capsule directly without any continuity solution (i.e. without any intermediate connector), in such a manner as to form a one-piece and fully self-contained or “autonomous” device;
said lead also being of the “micro-lead” type, i.e. a miniaturized lead of very small diameter (typically no larger than 1.5 French (0.5 mm), preferably no larger than 1 French (0.33 mm)) and without any internal lumen, and formed of a core cable covered with a layer of insulation with, in the distal region, one or more selectively exposed or bared zones forming detection/stimulation electrodes.

For example, such a hybrid capsule is described in detail in Document EP 2 959 828 A1 (SORIN CRM), which can be referred to for further details, the teaching of that document being fully applicable, as far as it is concerned, to the present invention.

The hybrid capsule 110 is associated with at least two leads 120, 130, namely a cardiostimulation lead 120 and a neurostimulation lead 130.

The lead 120 comprises a plurality of detection/stimulation electrodes 122, and more specifically four such electrodes in the example shown, that may be used independently from one another or not independently from one another (single-pole or multi-pole configuration, as applicable), for stimulating or for detecting. The possibility of moving the lead forwards or backwards in the catheter serving to implant it enables the position of the electrodes to be adjusted relative to the target zone and as a function of the results of the electrical testing performed during implanting.

Similarly, the lead 130 comprises a plurality of electrodes 132, and more specifically also four such electrodes in the example shown, that may be used independently from one another or not independently from one another (single-pole or multi-pole configuration, as applicable). It should be noted that to make the drawings clearer, these electrodes are symbolized by dots in the figures, but that, in practice, they do not give rise to any extra thickness in the lead at the locations at which they are situated, the leads 120 and 130 being leads of the “isodiametric” type procuring difficulty—free insertion and navigation in even the narrowest of vessels.

In the embodiment shown, but optionally, a third lead 140 is provided that is a neurostimulation and/or neurodetection lead that is also provided with a plurality of electrodes 142, in the same way as the first neurostimulation lead 130.

Each of the leads 120, 130, and optionally 140, is a lead of the “micro-lead” type as described in particular in EP 2 455 131 A1, EP 2 559 453 A1, EP 2 581 107 A1 and EP 2 719 422 A1 (Sorin CRM SAS).

The lead is of very small diameter in its distal portion, and typically has a diameter less than 1.5 French (0.5 mm), and preferably no larger than 1 French (0.33 mm). The very small diameter of the micro-cable makes it possible to use the entire length of the vein and to cannulate veins of very small diameter that are not used by usual techniques due to the excessive size of conventional coronary leads. As a result, it becomes possible to treat new regions that are difficult to reach, and thus to make optimum use of all of the veins present in the basal zone, in particular to avoid the risk of phrenic stimulation, which generally increases when the lead is too distal. The size of the lead body is a factor that is directly related to the capacities for guiding the lead in controlled manner in the coronary venous system, so that it is possible to select particular stimulation sites therein that are situated in certain collateral veins.

The micro-lead is made up of at least one cable of the “micro-cable” type. Essentially, a micro-cable comprises an electrically conductive core that is provided with an insulating covering, except at points that are exposed or bared and that serve to constitute detection/simulation electrodes. Preferably, a succession of a plurality of exposed or bared points are provided that, together, constitute a network or array of individual electrodes making it possible to increase the number of stimulation points in a target zone.

Advantageously, as described in above-mentioned EP 2 719 422 A1, a plurality of electrically independent micro-cables are assembled together, e.g. stranded, in such a manner as to obtain a micro-lead provided with a plurality of distinctly selectable electrodes (multi-pole configuration). This configuration offers the possibility of implementing an “electrical repositioning” function consisting in selecting, from among a plurality of stimulation points corresponding to a plurality of electrodes connected to respective micro-cables of the lead, that stimulation point that procures the best efficacy. Such selection may be effected equally well at the time at which the lead is implanted as subsequently, by conducting tests at regular intervals to check that the initially chosen site is still optimum, and possibly by selecting another site if it is not still optimum. In a variant, the micro-cable may however comprise a single conductor only, so that the exposed or bared regions form electrodes that, from an electrical point of view, are all active and electrically connected in parallel to the same potential (single-pole configuration).

The specific structure of the micro-cable is as described, in particular, in EP 2 455 131 A1 and EP 2 581 107 A1 (Sorin CRM), which documents can be referred to for further details. Those documents also give indications about the mode of operation for implanting the micro-cable into a coronary system.

As regards the materials that can be used, and in non-limiting manner, the core of the micro-cable is advantageously a multi-strand structure, in which each strand is preferably made of Nitinol (a nickel titanium (NiTi) alloy), or of MP35N-LT (35% Ni, 35% Co, 20% Cr and 10% Mo), the essential advantage of those materials being their extreme fatigue endurance, with coating with a sheath made of platinum-iridium alloy or of tantalum (for radio-opacity and biostability). Such a structure makes it possible to optimize the response to the requirements of corrosion resistance at the electrodes and of endurance to heart movements. Such micro-cables are available, for example, from Fort Wayne Metals Inc., Fort Wayne, USA. The layer of insulation, of thickness of about 25 micrometers (μm), is formed over the core, e.g. by co-extrusion or by heating a heat-shrinkable tube. The insulation may be a fine layer of Parylene in which openings are provided, e.g. by plasma ablation, for forming the electrodes, a polyurethane tube that is interrupted at the locations of the electrodes, or indeed one or more layers constituted by tubes made of polyethylene terephthalate (PET), a fluoropolymer, poly (methyl methacrylate) (PMMA), polyether ether ketone (PEEK), polyimide, or any other similar material that is appropriate.

The leads 120, 130, 140 are implanted in the left or right pericardiophrenic veins LPCPV or RPCPV by conventional techniques using a guidewire, access to the veins being achieved via the left subclavian vein (LSCV), which constitutes a common access for all of the micro-leads 120, 130, and 140. This access is a usual access that does not involve any modification in the operating techniques for the practitioner in charge of implementing all of the elements of the device.

The cardiostimulation lead 120 is implanted in the coronary venous system CVS so as to detect cardiac potentials at the left ventricle LV, and so as to perform any stimulation of that ventricle. Said lead is implanted by conventional techniques, with access being via the coronary sinus, and then an implantation catheter advancing into the venous system and positioning the active portion of the lead that carries the electrodes 122 at the chosen stimulation target zone.

The neurostimulation lead 130 is implanted in the left pericardiophrenic vein LPCPV in the upper portion, close to the left pericardiophrenic nerve LPCPN so as to stimulate said nerve (which is a motor nerve), thereby stimulating the diaphragm D in order to regulate the respiratory rhythm of the patient by applying a stimulation to this efferent pathway.

The lead 140 that acts as a neurodetection lead is implanted in the right pericardiophrenic vein RPCPV in the upper portion, close to the right pericardiophrenic nerve RPCPN, for detecting, on the nerve (which is a sensory nerve), nerve potentials coming from the diaphragm D, in such a manner as to obtain feedback signals via this afferent pathway, which signals can be used for accurately regulating the respiratory rhythm.

In a variant embodiment, the stimulation of the diaphragm D is a quasi-direct stimulation effected by means of a longer neurostimulation lead (the distal portion of which is shown in dashed lines at 130′) having its active portion 132′ inserted into one of the branched vessels of the left pericardiophrenic vein LPCPV in such a manner that said active portion finds itself positioned in the vicinity of the diaphragm D.

The lengths of the leads 120, 130, and 140 (e.g. as measured from the body of the hybrid capsule 110 to the respective most distal electrode 122, 132, or 142) are specifically adapted to the function that they are to perform, taking into account the distance between the hybrid capsule 110 of the target zone to be stimulated and/or the place where the electric potentials are to be collected, respectively the nerve electric potentials or the electric potentials for depolarizing the myocardium.

Thus, for stimulating the left pericardiophrenic nerve LPCPN via the left pericardiophrenic vein LPCPV in the upper portion, the length of the lead 130 typically lies approximately in the range 50 cm to 60 cm. For stimulating the right pericardiophrenic nerve RPCPN via the right pericardiophrenic vein RPCPV in the upper portion, the length of the lead 140 typically lies approximately in the range 55 cm to 65 cm.

In the above-mentioned variant for quasi-direct stimulation of the diaphragm D, those dimensions are increased by about 10 cm (i.e. in the range 60 cm to 70 cm for a lead 130′ for stimulating the diaphragm via one of the branched vessels of the left pericardiophrenic vein LPCPV.

In order to perform detection/stimulation for the right cavities of the heart, provision is advantageously made to use autonomous capsules 200, 300 of the leadless type in combination with the hybrid capsule device 100 of the invention, which leadless autonomous capsules are implanted in the right atrium RA and in the right ventricle RV.

Capsules of the leadless type are devices in the form of autonomous capsules that are implantable in a cardiac cavity (ventricle, atrium, or indeed arterial left cardiac cavity). Such capsules are devoid of any physical link or connection to a main device that is either implanted (such as a stimulation or pace-making pulse generator) or not implanted (an external peripheral such as a programmer or monitoring device for remotely monitoring the patient), and, for that reason, they are referred to as “leadless capsules” to distinguish them from electrodes that are disposed at the distal end of a conventional lead, through the entire length of which one or more conductors extend that connect the distal electrode metallically to a connector for connection to a pulse generator.

Leadless capsules 200 and 300 comprise a body provided at one of its ends with an anchor member, which is generally a projecting helical screw axially extending the body of the capsule, and designed to penetrate into the cardiac tissue by screwing at the implantation site, in the same way as for leads having conventional screws.

EP 2 394 695 A1 (Sorin CRM) describes such a type of leadless capsule having a screw.

Each of the hybrid or leadless capsules 100 (hybrid), 200 (leadless), 300 (leadless), and the hybrid capsule 110 comprises control electronic circuits coupled to wireless communication transmitter/receiver means to make mutual communications possible between the various capsules 110, 200, 300, and, when so desired, to make communications possible between the hybrid capsule 110 and external apparatus such as a programmer or monitoring equipment.

The hybrid capsule 110 may thus act as a master device or hub, in a star wireless network architecture, the endocardial leadless capsules 200 and 300 being the slave devices.

The above-described set, with the hybrid capsule 110 and the cardiostimulation lead 120 and the neurostimulation lead(s) 130, and optionally 140, and the ventricular leadless capsule 300, and, optionally, the atrial leadless capsule 200, makes it possible to apply combined cardio-respiratory therapy to the patient.

Thus, cardiac rhythm treatment, which is treatment of the conventional single-chamber, multi-chamber, or resynchronization type, is associated with central sleep apnea treatment, by stimulating the diaphragm via the pericardiophrenic nerve.

More specifically, the neurostimulation treatment is effected by:
stimulating the diaphragm D via the left pericardiophrenic nerve LPCPN, which receives pulses from the electrodes 132 of the lead 130 implanted in the left pericardiophrenic vein LPCPV in the vicinity of said nerve; and
detecting the activity fed back from the diaphragm D via the right pericardiophrenic nerve RPCPN, the variations in electric potentials of which are collected by the electrodes 142 of the lead 140 implanted in the right pericardiophrenic vein RPCPV close to said nerve, in the configuration in which such a lead 140 is present.

FIG. 3 is an enlarged view of the hybrid capsule 110 of the device of the invention.

This capsule is associated with the leads 120, 130, and 140 via a permanent simplified connection system that is devoid of any electrical connector: the leads 120, 130, 140 extend the body of the capsule 110 without any continuity solution due to the absence of any connector, with a transition intermediate region 150 procuring a progressive stiffness gradient between the rigid end of the body and the flexible portions of the micro-leads.

For example, the dimensions of the hybrid capsule 110 are approximately 10 mm×30 mm×10 mm. This capsule incorporates a low-consumption, typically from 5 microwatts (μW) to 8 μW, electronic architecture, powered by a battery or, in a variant, an energy harvester system. Advantageously, the electronic circuit of the hybrid capsule also incorporates one or more servo-control sensors, e.g. a 3D accelerometer and a thermistor making it possible to measure body temperature (in a configuration in which the body of the hybrid capsule 110 is implanted subcutaneously).

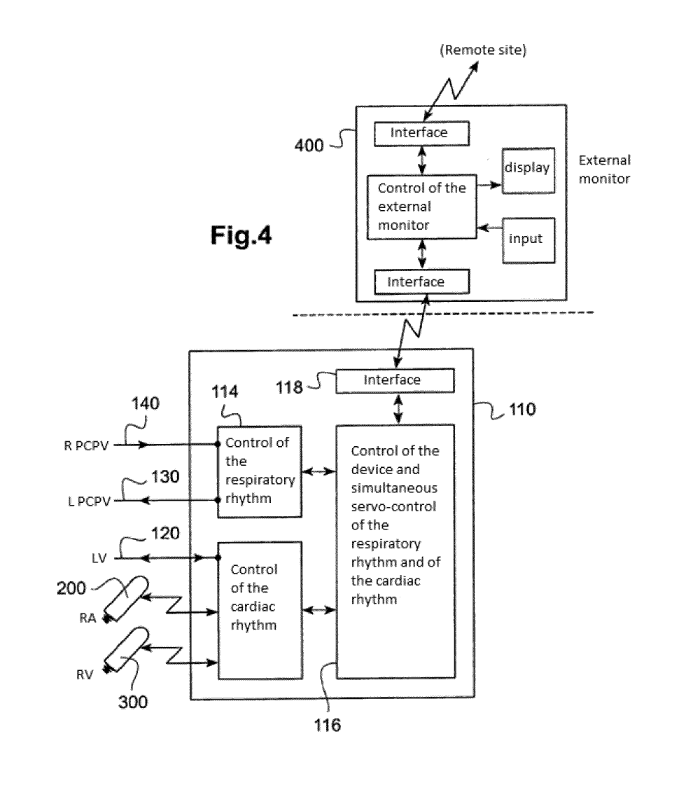

FIG. 4 is a block diagram showing the various elements of the system of the invention and the interactions between the various elements.

The hybrid capsule 110 comprises a cardiac rhythm control circuit 112, connected to the cardiostimulation lead 120 implanted in the left ventricle, and comprising wireless communications means for communicating with the leadless capsules 200 and 300 respectively implanted in the right atrium and in the right ventricle.

The hybrid capsule 110 also comprises a respiratory rhythm control circuit 114 connected to the cardiostimulation leads 130, 140 implanted respectively in the left and right pericardiophrenic veins.

The circuits 112 and 114 co-operate with a circuit 116 for controlling the device and for simultaneously servo-controlling the respiratory rhythm and the cardiac rhythm, controlled respectively by the circuits 114 and 112.

An interface 118 is also provided for communicating with external apparatus 400, which may, in particular, be a practitioner's programmer, the communication then serving to interrogate the implanted set, to read data stored in a memory in it, to modify certain parameters, etc. The external apparatus 400 may also be home monitoring apparatus, i.e. external apparatus for monitoring the state of the patient at home, optionally with it being possible to transmit information to a remote hospital or other site. Sorin CRM's Smartview Remote Monitoring System is an example of such external apparatus.

The communication between the hybrid capsule and the leadless capsules 200 and 300 is intra-body communication of the Human Body Communication (HBC) C) type, e.g. implementing a technique of communication by means of pulses transmitted via the interstitial tissue of the body of the patient, such pulses being generated, emitted, collected, and detected by appropriate circuits, e.g. such as those described in EP 2 441 491 A1 (Sorin CRM) and EP 2 486 953 A1 (Sorin CRM). The communication between the hybrid capsule 110 and the external apparatus 400 is radiofrequency (RF) telemetry communication, e.g. in the Medical Implant Communication System (MICS) band, in the Medical Data Service (MEDS) band, or in the Industrial, Scientific and Medical (ISM) band, or indeed using the Bluetooth protocol.

1-8. (canceled)

9. An active implantable medical device for combined treatment of cardiac rhythm and of respiratory rhythm, said device comprising:
an autonomous unit designed to be implanted subcutaneously, said autonomous unit comprising a sealed body receiving electronic circuits and an electrical power supply for powering said electronic circuits, the electronic circuits comprising:
a cardiac rhythm control circuit comprising a circuit configured to detect cardiac potentials and a generator configured to generate cardiostimulation pulses;
a respiratory rhythm control circuit comprising a circuit configured to detect nerve potentials and a generator configured to generate neurostimulation pulses; and
a combined control circuit configured to provide combined control of the cardiac rhythm control circuit and of the respiratory rhythm control circuit;
at least one cardiostimulation lead comprising, in a distal active portion, at least one detection or stimulation electrode suitable for being implanted in or on the myocardium of a patient wearing the device; and
at least one neurostimulation lead comprising, in a distal active portion, at least one detection or stimulation electrode suitable for being implanted on or close to a nerve of the patient wearing the device;
wherein the at least one cardiostimulation lead and the at least one neurostimulation lead are micro-leads each of which comprises at least one micro-cable formed of an electrically conductive core cable connected to a pole of said electronic circuits, with a layer of insulation surrounding the core cable, which is provided with at least one selectively bared or exposed zone provided in the layer of insulation and designed to form said detection and/or stimulation electrode, the diameter of the micro-leads being no more than 1 French (0.33 mm) in their most distal region including said selectively bared or exposed zone; and
said device is an autonomous hybrid capsule incorporating in the same set said autonomous unit, said at least one cardiostimulation lead and said at least one neurostimulation lead;
wherein the hybrid capsule is devoid of any electrical connector between the electronic circuits and the cardiostimulation and neurostimulation micro-leads;
and each of the at least one cardiostimulation lead and of the at least one neurostimulation lead extend the sealed body of the autonomous unit at one end thereof.

10. The device of claim 9, wherein the at least one cardiostimulation micro-lead or the at least one neurostimulation micro-lead is a multi-pole micro-lead comprising a plurality of said electrodes in its distal active portion.

11. The device of claim 9, wherein said cardiostimulation micro-lead is a stimulation micro-lead that is suitable for being implanted in the coronary venous system for stimulating a left cavity of the myocardium of the patient wearing the device.

12. The device of claim 9, wherein said neurostimulation micro-lead is configured to provide indirect stimulation of the diaphragm via the left pericardiophrenic nerve, wherein said neurostimulation micro-lead is a micro-lead suitable for being implanted in the left pericardiophrenic vein, the length between the body of the autonomous unit and the most distal electrode of the neurostimulation micro-lead being from 50 cm to 60 cm.

13. The device of claim 9, wherein said neurostimulation micro-lead is configured to provide direct stimulation of the diaphragm, wherein said neurostimulation micro-lead is a micro-lead suitable for being implanted in one of the branched vessels of the left pericardiophrenic vein, the length between the body of the autonomous unit and the most distal electrode of the neurostimulation micro-lead being from 60 cm to 70 cm.

14. The device of claim 9, further comprising:
a neurodetection micro-lead suitable for being implanted on or close to a nerve of an afferent pathway of the patient wearing the device;
said neurostimulation micro-lead being a micro-lead suitable for being implanted on or close to a nerve of an efferent pathway of the patient wearing the device.

15. The device of claim 14, wherein said neurodetection micro-lead is a micro-lead suitable for being implanted in the right pericardiophrenic vein, the length between the body of the autonomous unit and the most distal electrode of the neurodetection micro-lead being from 55 cm to 65 cm.

16. The device of claim 9, wherein the electronic circuits of the autonomous unit further comprise a transmitter/receiver for mutual wireless intra-body with intra-body autonomous leadless capsules suitable for being implanted in or on the myocardium of the patient, and an interface for enabling said transmitter/receiver to interface with the cardiac rhythm control circuit.
